(19) United States
(12) Patent Application Publication
(10) Pub. No.: US 2017/0333680 A1
(43) Pub. Date: Nov. 23, 2017

(54) CENTRAL VENOUS CATHETER HUB COCOON

(71) Applicants: Anthony Bentley, Seabrook, TX (US); Ingrid Parry, Carmichael, CA (US); Scott Naples, Hudson, OH (US); Heather McKelvy, Menlo Park, CA (US); Tammy Hoy, League City, TX (US); Karla Abela, Pearland, TX (US)

(72) Inventors: Anthony Bentley, Seabrook, TX (US); Ingrid Parry, Carmichael, CA (US); Scott Naples, Hudson, OH (US); Heather McKelvy, Menlo Park, CA (US); Tammy Hoy, League City, TX (US); Karla Abela, Pearland, TX (US)

(21) Appl. No.: 15/602,635

(22) Filed: May 23, 2017

Related U.S. Application Data
(60) Provisional application No. 62/339,995, filed on May 23, 2016.

(51) Int. Cl. A61M 25/02; A61M 39/18; A61M 25/06; A61M 39/16

(52) U.S. Cl. CPC A61M 25/02; A61M 39/165; A61M 39/18; A61M 25/0606; A61M 2025/024; A61M 2025/028; A61M 2025/0206

(57) ABSTRACT

Presented herein is a catheter protection device having a first housing portion and a second housing portion, the first housing portion and the second housing portion defining a hollow interior therebetween. A latching mechanism is configured to releasably secure the second housing portion to the first housing portion. A first port is defined in the (i) first housing portion, (ii) the second housing portion, or (iii) both the first housing portion and the second housing portion, the first port having a first gasket disposed at the first port. A second port is defined in the (i) first housing portion, (ii) the second housing portion, or (iii) both the first housing portion and the second housing portion, the second port having a second gasket disposed at the second port. An absorbent material is disposed in the hollow interior and includes a concentration of an antimicrobial substance.

### CROSS-REFERENCE TO RELATED APPLICATIONS

This application claims the benefit of U.S. Provisional Patent Application No. 62/339,995, filed May 23, 2016, which is incorporated herein by reference in its entirety.

### TECHNICAL FIELD

The present disclosure relates to catheter protection devices. Specifically, the present disclosure relates to a cocoon for enclosing access ports on a central venous catheter.

### BACKGROUND

Catheters and other devices configured to be inserted into a patient's body can easily become contaminated and lead to infection. In particular, central venous catheters—which have one end left disposed in a patient's vein with another end open to the air—often stay connected to patients for long periods of time. The central venous catheter ends in a hub that has a port open to the air is for connecting syringes, IV's, auxiliary lines, or anything else needed to provide medicine. When the central venous catheter is not in use, the hub is not connected to anything and can easily become contaminated and lead to infections. Current protective devices inadequately protect the catheter hub from contamination and the patient from infection. Aspects of the present disclosure provide a new catheter protection device that solves this problem and other problems.

### SUMMARY

According to aspects of the present disclosure, a protective device for a catheter hub comprises a first housing portion and a second housing portion, the first housing portion and the second housing portion defining a hollow interior therebetween. The device further comprises a latching mechanism configured to releasably secure the second housing portion to the first housing portion; a first port defined in the (i) first housing portion, (ii) the second housing portion, or (iii) both the first housing portion and the second housing portion, the first port having a first gasket disposed at the first port; a second port defined in the (i) first housing portion, (ii) the second housing portion, or (iii) both the first housing portion and the second housing portion, the second port having a second gasket disposed at the second port; and an absorbent material disposed in the hollow interior, the absorbent material including a concentration of an antimicrobial substance.

According to further aspects of the present disclosure, a method of protecting a catheter hub comprises providing a catheter protection device; disposing the catheter hub within a hollow interior of the catheter protection device while the catheter protection device is in an open state; transitioning the catheter protection device from the open state to a closed state; and releasably securing a first housing portion of the catheter protection device to a second housing portion of the catheter protection device.

According to still further aspects of the present disclosure, a protective device for a catheter hub comprises a housing having a hollow interior; a latching mechanism configured to releasably secure the housing in a closed state; a plurality of ports defined in the housing; a plurality of gaskets, each gasket being disposed at one of the plurality of ports; and a concentration of an antimicrobial substance in the hollow interior of the housing.

These and other capabilities of the invention, along with the invention itself, will be more fully understood after a review of the following figures, detailed description, and claims.

### BRIEF DESCRIPTION OF THE DRAWINGS

The disclosure will be better understood from the following description of exemplary embodiments together with reference to the accompanying drawings.

FIG. 1 shows a perspective view of an embodiment of a catheter protection device in an open state, in accord with aspects of the present disclosure.

FIG. 2 shows a perspective view of the catheter protection device of FIG. 1 with a hub disposed therein and a line extending out of the catheter protection device, in accord with aspects of the present disclosure.

FIG. 3 shows a perspective view of the catheter protection device of FIG. 1 with a hub disposed therein and multiple lines extending out of the catheter protection device, in accord with aspects of the present disclosure.

FIG. 4 shows a perspective view of the catheter protection device of FIG. 1 in a closed state, in accord with aspects of the present disclosure.

FIG. 5 shows a perspective view of another embodiment of a catheter protection device, in accord with aspects of the present disclosure.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.” Additionally, the singular terms “a,” “an,” and “the” include plural referents unless context clearly indicates otherwise.

A catheter protection device according to aspects of the present disclosure may be used to help prevent contamination of a central venous catheter. A central venous catheter (or central line) generally comprises tubing that is inserted into a large vein of a patient, such as the jugular vein, the subclavian vein, or the femoral vein. One open end of the catheter is disposed in the patient's vein, and the other open end of the catheter is disposed outside the body and can be open to the air. This end can generally have a port, hub, manifold, or similar structure connected thereto that allows for a syringe, IV bag, other tubing, or some other apparatus to be connected to the catheter in order to deliver fluid to the patient's vein. As used herein, “central line” refers to the tubing that is inserted into the patient and has an open-air port at the other end; “auxiliary line” refers to any device or other tubing that may be connected to the port; and “hub” refers to the structure that is disposed at the end of the central line and that the auxiliary line is coupled to.

Sometimes, the hub can be left open to the air, which can lead to what is known as central line-associated bloodstream infections (CLABSI), which can cause injury to patients and can even lead to death, and can easily increase healthcare costs. The catheter protection device can envelop the hub and establish or maintain an asceptic environment within a hollow interior of the catheter protection device where the hub is disposed during use of the catheter protection device. The catheter protection device allows the central line and any auxiliary lines access to the port. The catheter protection device can also be easily removed to gain direct access to the hub. The interior of the catheter protection device may be lined with an antimicrobial substance to further protect the hub and maintain the asceptic environment around the hub. When the central venous catheter is not in use, i.e. there is no auxiliary line coupled to the hub, the hub can be left open to the air.

While the present disclosure generally refers to a central venous catheter, any system providing access to a patient's body can be suitable for use with the catheter protection device. For example, the catheter protection device may be used with peripherally inserted central catheters (PICC lines), uretic catheters such as Foley catheters, normal IV tubing, and feeding tubs such as a gastric tube (G-Tube) or a nasogastric tube (NG-Tube). Other types of catheters or tubes are also contemplated for use with the catheter protection device of the present disclosure.

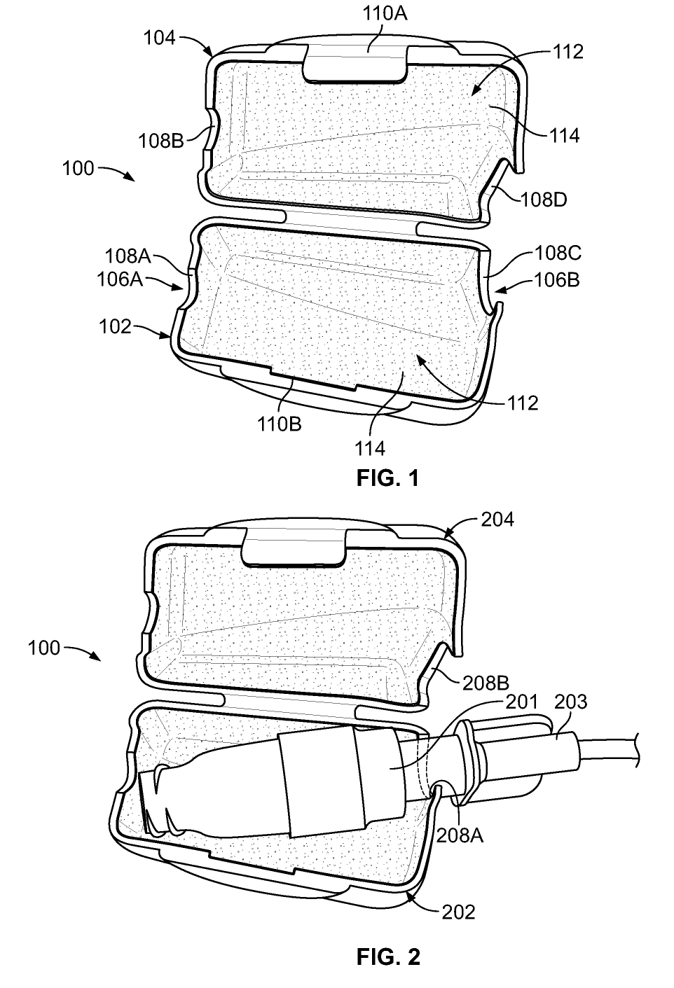

Referring now to FIG. 1, a catheter protection device 100 in an open state is illustrated. The housing of the catheter protection device 100 comprises a first housing portion 102 and a second housing portion 104. In an embodiment illustrated in FIG. 1, the first housing portion 102 and the second housing portion 104 are rotationally coupled in a clamshell configuration. In the clamshell configuration, the second housing portion 104 is rotationally coupled to the first housing portion 102 to allow the catheter protection device 100 to transition between the open state and a closed state. In the closed state, the second housing portion 104 covers the first housing portion 102, and the first housing portion 102 and the second housing portion 104 form a hollow interior therebetween in which the catheter hub may be disposed. The housing of the catheter protection device 100 may comprise a rigid polymer, a flexible polymer, foam, paper, or any other suitable material.

The catheter protection device 100 may include a latching mechanism to secure the catheter protection device in the closed state, i.e. releasably secure the second housing portion 104 to the first housing portion 102. In the embodiment illustrated in FIG. 1, the latching mechanism includes a structure 110A configured to mate with a groove or depression 110B. The latching mechanism could also be a bar and hook latching mechanism, a ball and detent latching mechanism, or any other suitable latching mechanism. The latching mechanism is generally a quick-latching mechanism that can be operated with one hand, and does not require a user to contact the hub, the central line, the auxiliary line, or the interior of the catheter protection device when placing the catheter protection device on the hub or removing the catheter protection deice from the hub. However, the latching mechanism is generally such that small children are deterred from opening the catheter protection device and removing the hub.

In the embodiment illustrated in FIG. 1, the first housing portion 102 and the second housing portion 104 are arranged in a clamshell design where the second housing portion 104 is rotationally coupled to the first housing portion 102. The second housing portion 104 may be rotationally coupled to the first housing portion 102 via a hinge. In this embodiment, both the first housing portion 102 and the second housing portion 104 include structures extending along corresponding edges of the first housing portion 102 and the second housing portion 104, each of the structures including an aperture. A rod or pin may then be inserted through the apertures such that the rod or pin is parallel to the corresponding edges of the first housing portion 102 and the second housing portion 104, thus allowing the rotational movement between the first housing portion 102 and the second housing portion 104. In another embodiment, either the first housing portion 102 or the second housing portion 104 may include a pair of pins or similar structures extending from opposing edges of the second housing portion 104. The pair of pins may then be inserted into depressions defined along opposing edges of the first housing portion 102 or the second housing portion 104. In yet another embodiment, the first housing portion 102 and the second housing portion 104 may be connected to each other via a living hinge. Other embodiments of the clamshell design are contemplated that allow rotational movement between the first housing portion 102 and the second housing portion 104.

In another embodiment of the catheter protection device, the first housing portion and the second housing portion are arranged in a tray-and-lid design. In this design, the second housing portion is placed onto the first housing portion to form the hollow interior into which the hub is disposed during use. In this embodiment, the latching mechanism could include a single latching mechanism, or could include multiple latching mechanisms on multiple corresponding edges of the first and second housing portion. The latching mechanism could also include an extending structure defined along at least one edge of the first housing portion and a corresponding groove defined along at least one edge of the second housing portion. The extending structure and the corresponding groove are configured to mate with each other when the second housing portion is placed onto the first housing portion to releasably secure the second housing portion to the first housing portion.

A further embodiment of the catheter protection device has a split clamshell design. This embodiment includes a third housing portion that is rotationally coupled to the first housing portion. In this embodiment, both the second housing portion and the third housing portion are rotated down to cover the first housing portion when the catheter protection device is in the closed state. This embodiment may include a single latching mechanism configured to releasably secure both the second housing portion and the third housing portion to the first housing portion, or multiple latching mechanism, each configured to secure the second housing portion, the third housing portion, or both the second housing portion and the third housing portion to the first housing portion. In this embodiment, the second housing portion and the third housing portion may be of approximately the same size and thus can each cover about half of the first housing portion, such that the catheter protection device has separate opening halves. In other embodiments, the second housing portion and the third housing portion have different sizes.

In yet a further embodiment, the catheter protection device has a split tray-and-lid design. This embodiment includes a third housing portion that can be placed onto the first housing portion. In this embodiment, both the second housing portion and the third housing portion cover the first housing portion to form the hollow interior of the catheter protection device where the hub will be disposed when in use. The second housing portion and the third housing portion may be releasably secured to the first housing portion in the same manner as in the tray-and-lid design. In this embodiment, the second housing portion and the third housing portion may be of approximately the same size and thus may each cover about half of the first housing portion, such that the catheter protection device has separate opening covers.

Referring again to FIG. 1, the catheter protection device 100 may include one or more ports 106A, 106B defined in the housing of the catheter protection device 100. The ports 106A, 106B may be defined in (i) the first housing portion 102; (ii) the second housing portion 104; or (iii) both the first housing portion 102 and the second housing portion 104. These ports may be formed by cut-out portions 108A, 108C formed in the first housing portion 102 and cut-out portions 108B, 108D formed in the second housing portion 104. When the catheter protection device 100 is in use and in the closed state, cut-out portion 108A and cut-out portion 108B are aligned such that they form port 106A. Similarly, cut-out portion 108C and cut-out portion 108D are aligned such that they form port 106B. Alternatively, ports 106A, 106B may be defined wholly in either the first housing portion 102 or the second housing portion 104. Embodiments of the catheter protection device 100 having a third housing portion may have one or more ports defined in (i) the first housing portion; (ii) the second housing portion; (iii) the third housing portion; (iv) both the first housing portion and the second housing portion; (v) both the first housing portion and the third housing portion; (vi) both the second housing portion and the third housing portion; or (vii) the first housing portion, the second housing portion, and the third housing portion.

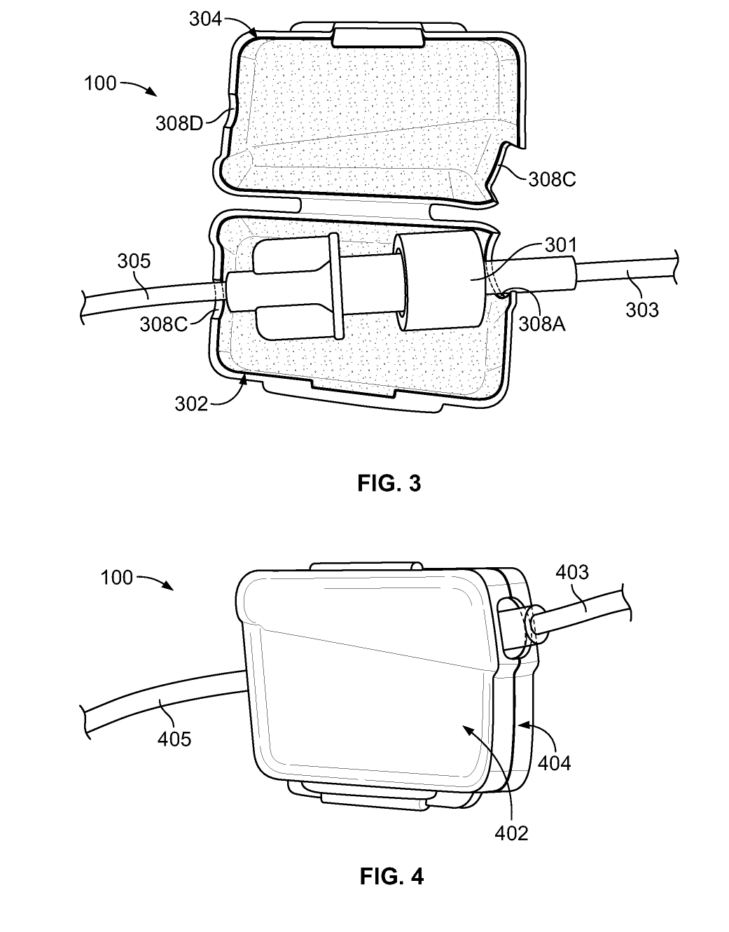

Each port is sized such that tubing, such as the central line or any auxiliary lines, can be disposed through the port and connected to the hub disposed in the hollow interior of the catheter protection device without crimping the tubing. The ports thus allow the catheter protection device 100 to be utilized with a central venous catheter while the central venous catheter is being used to deliver fluid or medicine to the patient without impeding fluid flow through the central line or the auxiliary lines in any manner. As illustrated in FIG. 1, the catheter protection device 100 may generally have at least two ports 106A, 106B defined in the housing thereof. The central line may be inserted through port 106A, while the auxiliary line may be inserted through port 106B (see FIG. 3). Other embodiments are contemplated having any number of ports, depending upon the application for which the catheter protection device 100 is being utilized. In another embodiment, the catheter protection device 100 includes only one port.

Each port can have a gasket disposed at the port to both cushion the tubing when inserted through the port and to effectively seal off the port when the port is not in use. The gasket is generally made from a soft conformable material such as foam or a soft elastomer. Other embodiments are contemplated that utilize other suitable materials. The gasket may be disposed about the periphery of the port to cushion the contact between the periphery of the port and the tubing. The gasket may also include a number of bristles or fingers that contact each other near the port and effectively seal the port, but still allow the tubing to be inserted into the catheter protection device 100. The seal acts as a dust and dirt barrier, and generally protects the interior of the catheter protection device from contamination when the port is not being used. The seal is also splash-resistant and prevents any undesired fluid from entering the catheter protection device 100 through the port. Thus, the gaskets disposed at the ports eliminate the requirement that the catheter protection device 100 must be removed from the hub for an auxiliary line to be coupled to the hub. This reduces the chances of the patient contracting a blood-stream infection, and increases the usefulness and efficiency of the catheter protection device 100.

The catheter protection device 100 can also include an absorbent material 112 that lines the interior of the catheter protection device 100. This absorbent material 112 may comprise non-woven fabric, woven fabric, a semi-solid gel, foam, or any other suitable material. The absorbent material 112 is impregnated with a sufficient concentration of an antimicrobial substance 114 to remove or destroy any contaminants present at the hub when the hub is inserted into the catheter protection device 100, and to establish an asceptic environment within the hollow interior of the catheter protection device 100. The combination of the absorbent material 112 and the antimicrobial substance 114 can then maintain the asceptic environment with the hollow interior of the catheter protection device. The antimicrobial substance 114 can comprise isopropyl alcohol, ethyl alcohol, a chlorhexidine gluconate solution, a providone-iodine solution, a hydrogen peroxide solution, a sodium hypochlorite solution, a nano-silver suspension, or any combination of the above.

Referring now to FIG. 2, the catheter protection device 100 of FIG. 1 having a hub 201 disposed therein and a line 203 extending out of the catheter protection device 100 is illustrated. As shown, line 203 extends through cut-out portion 208A of the first housing portion 202. Line 203 may be a central line or an auxiliary line. A second cut-out portion 208B is located on the second housing portion 204 such that when the second housing portion 204 is rotated down and releasably secured to the first housing portion 202, the central line 203 will be disposed through the port.

Referring now to FIG. 3, the catheter protection device 100 of FIG. 1 having a hub 3012 disposed therein and multiple lines extending out of the catheter protection device 100 is illustrated. As shown, a first line 303 extends through cut-out portion 308A of the first housing portion 302, while a second line 305 extends through cut-out portion 308C of the first housing portion 302. In an embodiment, first line 303 is a central line and second line 305 is an auxiliary line. In another embodiment, first line 303 is an auxiliary line and second line 305 is a central line. Further cut-out portions 308B, 308D are defined in the second housing portion such that when the second housing portion 304 is rotated down and releasably secured to the first housing portion 202, the central line 303 and the auxiliary line 305 will be disposed through the ports formed in the catheter protection device 100.

Referring now to FIG. 4, the catheter protection device 100 in the closed state is illustrated. As shown, the second housing portion 404 is releasably secured to the first housing portion 402 with the hub disposed within the hollow interior of the catheter protection device 100. Both a first line 403 and a second line 405 are connected to the hub and extend out of the ports defined in the housing of the catheter protection device 100. In an embodiment, first line 403 is a central line and second line 405 is an auxiliary line. In another embodiment, first line 403 is an auxiliary line and second line 405 is a central line.

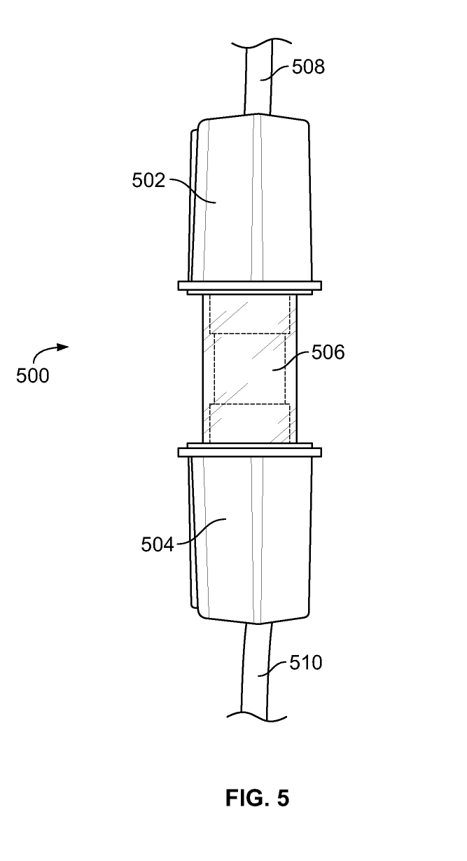

Referring now to FIG. 5, a catheter protection device 500 having a tray-and-lid design is illustrated. The catheter protection device 500 has a first housing portion 502 and a second housing portion 504. As shown, the first housing portion 502 and the second housing portion 504 are configured to be releasably coupled to each other when the first housing portion 502 and the second housing portion 504 are made to contact each other. Hub 506 will then be disposed in the hollow interior of the catheter protection device 500. Tubing 508 is shown extending out of the port defined in the first housing portion 502, while tubing 510 is shown extending out of the port defined in the second housing portion 504. In an embodiment, tubing 508 is a central line and tubing 510 is an auxiliary line. In another embodiment, tubing 508 is an auxiliary line and tubing 510 is a central line.

In an embodiment, the catheter protection device is a single-use device that is replaced or disposed of after it has been opened to access the hub. In another embodiment, the catheter protection device is used until the device or the hub becomes contaminated, at which point the catheter protection device may be replaced and disposed of. The catheter protection device itself can have a mechanism to indicate when it has become contaminated, or a person may use an external apparatus or method to determine when the catheter protection device has been contaminated. Prior to use, the catheter protection device is generally sterile and is contained in sterile packaging.

The catheter protection device can include an attachment mechanism to secure the catheter protection device itself to the patient or other object to stabilize the hub and ensure the catheter protection device does not come into contact with the ground. The attachment mechanism can be, for example, velcro, a snap fastener, a band, a clamp, a clip, or any other suitable attachment mechanism.

The catheter protection device can occupy a variety of different volumes. Alternatively, the hollow interior of the catheter protection device can occupy a variety of different volumes. For example, in an embodiment, the catheter protection device is sized such that a hub with a single open-air port for a single auxiliary line may fit snugly within the hollow interior of the catheter protection device when in the closed state. In another embodiment, the catheter protection device is sized to accommodate a hub having multiple open-air ports for auxiliary lines.

To use the catheter protection device, the catheter hub is disposed within the hollow interior of the catheter protection device. The catheter protection device is then transitioned from the open state to the closed state. In the clamshell design, this is caused by rotating the second housing portion down to cover the first housing portion. In the split clamshell design, this is caused by rotating the second housing portion and the third housing portion down to cover the first housing portion. In the tray-and-lid design, this is caused by placing the second housing portion on top of or against first housing portion to form the hollow interior. In the split tray-and-lid design, this is caused by placing the second housing portion and the third housing portion on top of or against the first housing portion to form the hollow interior. Generally the hub will have at least the central line coupled to the hub when the hub is placed into the hollow interior. In an embodiment, the hub is placed such that the central line will be disposed through a port defined in the catheter protection device when the catheter protection device is transitioned to the closed state. In another embodiment, the central line is inserted through the port and coupled to the hub after the catheter protection device has been transitioned to the closed state. In some embodiments, an auxiliary line may already be coupled to the hub when the hub is placed into the catheter protection device. In this embodiment, the hub is placed such that the auxiliary line will be disposed through a port defined in the catheter protection device when the catheter protection device is transitioned to the closed state. In still further embodiments, the auxiliary line is inserted through the port and coupled to the hub after the catheter protection device has been transitioned to the closed state. The catheter protection device is then releasably secured in the closed state by activating a latching mechanism to releasably secure the first housing portion to the second housing portion.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

What is claimed is:

1. A protective device for a catheter hub, the device comprising:
a first housing portion;
a second housing portion, the first housing portion and the second housing portion defining a hollow interior therebetween;
a latching mechanism configured to releasably secure the second housing portion to the first housing portion;
a first port defined in the (i) first housing portion, (ii) the second housing portion, or (iii) both the first housing portion and the second housing portion, the first port having a first gasket disposed at the first port;
a second port defined in the (i) first housing portion, (ii) the second housing portion, or (iii) both the first housing portion and the second housing portion, the second port having a second gasket disposed at the second port; and
an absorbent material disposed in the hollow interior, the absorbent material including a concentration of an antimicrobial substance.

2. The device of claim 1, wherein an edge of the second housing portion is rotationally coupled to an edge of the first housing portion, the edge of the first housing portion being adjacent to the edge of the second housing portion.

3. The device of claim 2, wherein the edge of the second housing portion is rotationally coupled to the adjacent edge of the first housing portion via a hinge.

4. The device of claim 1, further comprising a third housing portion coupled to the first housing portion.

5. The device of claim 4, wherein an edge of the third housing portion is rotationally coupled to a second edge of the first housing portion, the second edge of the first housing portion being adjacent to the edge of the third housing portion.

6. The device of claim 4, wherein the latching mechanism is further configured to releasably secure the third housing portion to the first housing portion.

7. The device of claim 4, further comprising a second latching mechanism, the second latching mechanism being configured to releasably secure the third housing portion to the first housing portion.

8. The device of claim 1, wherein at least one of the first gasket or the second gasket comprises foam or elastomer.

9. The device of claim 1, wherein the first port and the second port are sized to allow the catheter to be inserted through the first port or the second port without crimping the catheter.

10. The device of claim 1, further comprising an attachment mechanism configured to secure the device to an object.

11. The device of claim 10, wherein the attachment mechanism comprises velcro, a snap fastener, a band, a clamp, a clip, or any combination thereof.

12. The device of claim 1, wherein the concentration of the antimicrobial substance is configured to establish maintain asceptic conditions within the hollow interior of the device.

13. The device of claim 1, wherein the latching mechanism includes a structure defined along the edge of the first housing portion and a corresponding groove defined in the edge of the second housing portion, the structure of the second housing portion being configured to mate with the corresponding groove of the second housing portion.

14. A method of protecting a catheter hub, the method comprising:
providing a catheter protection device;
disposing the catheter hub within a hollow interior of the catheter protection device while the catheter protection device is in an open state;
transitioning the catheter protection device from the open state to a closed state; and
releasably securing a first housing portion of the catheter protection device to a second housing portion of the catheter protection device.

15. The method of claim 14, wherein a central line is coupled to the catheter hub, and wherein the central extends out of the catheter protection device when the first housing portion is of the catheter protection device is releasably secured to the second housing portion of the catheter protection device.

16. The method of claim 14, wherein the catheter protection device includes a concentration of an antimicrobial substance disposed within the hollow interior of the catheter protection device, the concentration of the antimicrobial substance being configured to establish or maintain an asceptic environment within the catheter protection device.

17. A protective device for a catheter hub the device comprising:
a housing having a hollow interior;
a latching mechanism configured to releasably secure the housing in a closed state;
a plurality of ports defined in the housing;
a plurality of gaskets, each gasket being disposed at one of the plurality of ports; and
a concentration of an antimicrobial substance in the hollow interior of the housing.

18. The protective device of claim 17, wherein the housing comprising a first housing portion and a second housing portion.

19. The protective device of claim 18, wherein the first housing portion and the second housing portion are arranged in a clamshell design.

20. The protective device of claim 18, wherein the first housing portion and the second housing portion are arranged in a tray-and-lid design.
